(19) United States
(12) Patent Application Publication
(10) Pub. No.: US 2015/0057342 A1
(43) Pub. Date: Feb. 26, 2015

(54) COMPOSITIONS FOR COMBINED IMMEDIATE AND SUSTAINED RELEASE OF CANNABINOIDS, METHODS OF MANUFACTURE AND USE THEREOF

(71) Applicant: CANNABICS PHARMACEUTICALS INC, Bethesda, MD (US)

(72) Inventors: Zohar KOREN, Gan Ner (IL); Eyal BALLAN, Ramat Hasharon (IL); Itamar BOROCHOV, Caesarea (IL); Shay Avraham SARID, GivatChen (IL)

(73) Assignee: CANNABICS PHARMACEUTICALS INC

(21) Appl. No.: 14/447,632

(22) Filed: Jul. 31, 2014

Related U.S. Application Data
(60) Provisional application No. 61/868,100, filed on Aug. 21, 2013.

(51) Int. Cl. A61K 9/48; A61K 31/047; A61K 9/20; A61K 31/343; A61K 31/085; A61K 31/352; A61K 31/353

(52) U.S. Cl. CPC A61K 9/4875; A61K 31/352; A61K 31/047; A61K 31/353; A61K 31/343; A61K 31/085; A61K 9/4858; A61K 9/4833; A61K 9/2095
USPC 514/454; 514/729; 514/733; 514/456; 514/468; 514/453

(57) ABSTRACT

Provided are oral pharmaceutical compositions comprising sustained release or a combination of sustained and immediate release formulation of cannabinoids, a process for their preparation and methods of use thereof.

### FIELD OF THE INVENTION

The present disclosure relates to oral pharmaceutical compositions comprising sustained release or a combination of sustained and immediate release formulation of cannabinoids, a process for their preparation and methods of use thereof.

### BACKGROUND OF THE INVENTION

The medicinal and psychoactive properties of the cannabis plant have been known for centuries. At present, cannabis is not legally available in most countries. However, there is growing legalization of its use, especially for medicinal purposes.

Years of research have failed to show that cannabis is dangerous and in fact, data prove the contrary. Cannabis has been shown to be safer, with fewer serious side effects, than most prescription drugs currently used as anti-emetics, muscle relaxants, hypnotics and analgesics, and the like. A disadvantage in treating patients with cannabis is the psychoactive effect, especially in “naive” cannabis users. Furthermore, there have been reports of unpleasant reactions to cannabis, such as anxiety, panic or hallucinations. It is believed that the undesirable side effects are most commonly associated with higher doses of cannabis, and are related to the difficulty in controlling the dosage when the drug is smoked or eaten in cannabis-enriched confectionaries.

Cannabis has been reported to benefit patients suffering from a wide range of symptoms experienced in connection with serious medical conditions. For example, cannabis has been used to alleviate symptoms associated with cancer, anorexia, AIDS, chronic pain, muscle spasticity, glaucoma, arthritis, migraine and many other illnesses. Cannabis is recognized as having anti-emetic properties and has been successfully used to treat nausea and vomiting in cancer patients undergoing chemotherapy. Studies also report use of cannabis in treating the weight loss syndrome of AIDS and for the treatment of glaucoma by reducing intraocular pressure. Furthermore, cannabis is known for its muscle relaxing and anti-convulsant effects.

The most prevalent mode of administration of medical cannabis is by smoking. Unfortunately, this mode of administration has adverse effects on the lungs. Cannabis smoke carries more tar and other particulate matter than tobacco, and may be a cause of lung diseases including lung cancer. Furthermore, many patients find the act of smoking unappealing, as well as generally unhealthy. It is known that some of the chemicals produced by smoking cannabis are aggressive and smoking has been shown to cause the gradual dissolving of teeth. For at least these reasons, smoking is a less desirable mode of administration for drugs, including cannabis.

U.S. Patent Application No. 2012/0231083 discloses an oral formulation of cannabis useful for treating sleep apnea.

There remains an unmet need for a measurable, reproducible and sustained release oral dosage form of cannabinoid for the treatment of multiple clinical conditions.

### SUMMARY OF THE INVENTION

The present inventors have developed oral formulations for sustained release and a combination of immediate and sustained release of cannabinoids that provide a long duration of activity, have a broad therapeutic window and obviate the undesirable side effects associated with smoking and ingesting foods with cannabis.

The advantages of oral compositions of cannabis include provision of an oral dose that obviates the need to smoke cannabis with the attendant hallucinatory effects. A sustained release formulation precludes the need for frequent administration of the drug while maintaining a uniform and constant release rate of the active pharmaceutical ingredient (API) over an extended period of time; thereby maintaining a stable and effective plasma drug concentration over time.

The present inventors have developed oral compositions of cannabinoids that provide immediate release, sustained release or a combination of immediate release and sustained release. The cannabinoid is easily prepared and formulated to provide therapeutically effective dosage forms of cannabis that are easily administered with high patient compliance.

The present inventors have unexpectedly discovered that an oral composition which includes a cannabinoid formulated with one or more emulsifiers provides a therapeutically effective sustained release formulation which delivers a long therapeutic effect of the API. The further inclusion of an oil based cannabinoid formulation to the dosage form provides a therapeutically effective immediate release and sustained release composition. The oil based cannabinoid immediate release portion of the composition is responsible for the quick onset of the therapeutic effects of the dosage form, for example, within 30-40 minutes, while the sustained release formulation is responsible for the gradual and long lasting therapeutic effects due to a constant and steady release of active cannabinoids from the gastric tract to the blood circulation.

Accordingly, in one aspect provided is a an oral composition of cannabinoids comprising a range of 0% to 99% immediate release fraction and a range of 100% to 1% sustained release fraction, wherein each fraction independently comprises a cannabinoid and an excipient; wherein the excipient in the immediate release fraction is an edible oil and the excipient in the sustained release fraction comprises an emulsifier.

In some embodiments, the cannabinoid is a lipophilic mixture of cannabinoids or a cannabinoid extract that contains at least one of the following: Tetrahydrocannabinol (THC), Cannabidiol (CBD), Cannabigerol (CBG), Cannabichromene (CBC), Cannabinol (CBN), Cannabielsoin (CBE), iso-Tetrahydrocannabimol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV) and Cannabigerol Monomethyl Ether (CBGM) and derivatives thereof. The cannabinoid may be natural or synthetic. In some embodiments, the cannabinoid(s) is present in a range of 1 mg to 350 mg active cannabinoid, preferably 5 mg to 100 mg, or 6 mg to 50 mg.

The sustained release fraction of the composition includes at least one emulsifier, selected from the group consisting of a monoglyceride, a diglyceride, beeswax, lecithin, a carrageenan and any mixture thereof. In some embodiments, the emulsifier includes a mixture of monoglycerides and diglycerides. In some embodiments, the emulsifier is a carrageenan, for example one or more of a lambda-carrageenan, kappa-carrageenan, iota-carrageenan and any mixture of the carrageenans. In certain preferred embodiments, the emulsifier includes a mixture of monoglycerides and diglycerides and a carrageenan. The emulsifier is present in a concentration range of 1% to 99% w/w, 5% to 80% w/w, 10% to 35%, 10% to 20% w/w, or about 15%-25% w/w. For example, the sustained release fraction includes an emulsifier which is a mixture of monoglyceride and diglyceride at a total concentration of 1% to 99% w/w and a carrageenan or mixture of carrageenans at a total concentration of 0.5% to 10% w/w.

In some embodiments, the sustained release fraction includes an emulsifier which is a mixture of monoglyceride and diglyceride for a total concentration of about 90% to 99% w/w. In various embodiments, the sustained release fraction includes an emulsifier which is a mixture of monoglyceride and diglyceride (about 70% to 79% w/w), and lecithin (about 20%) for a total concentration of about 90% to 99% w/w. In various embodiments, the sustained release fraction includes an emulsifier which is a mixture of monoglyceride and diglyceride (about 15% w/w), a carrageenan or mixture of carrageenans (about 1% w/w) and optionally lecithin (about 0% to 5%) for a total concentration of about 16% to 21% w/w. In some embodiments, the sustained release fraction further comprises an edible oil.

In various embodiments, the edible oils in the immediate release fraction and in the sustained release fraction, when present, are independently selected from coconut oil, wheat germ oil, olive oil, sprouted wheat oil, sesame oil, peanut oil, grape seed oil, palm oil, papaya seed oil or any combination thereof. In some embodiments, the edible oil in the immediate release fraction comprises coconut oil, preferably organic coconut oil in a concentration of about 50% to 99.9% w/w, or about 60%-98%, or about 90%-98% and optionally another edible oil or mixture of edible oils. When present, the edible oil in the sustained release fraction comprises coconut oil or a mixture of coconut oil and wheat sprout oil, preferably organic coconut oil and organic wheat sprout oil at a total concentration of about 50-80% or about 70 to 80%.

In some embodiments, the composition further comprises at least one excipient selected from the group consisting of a diluent, a binder, a lubricant, a disintegrant, a flavoring agent, a coloring agent, a stabilizer, a surfactant, a glidant, a plasticizer, a preservative, an essential oil and a sweetener. In various embodiments, the composition comprises an essential oil or a mixture of essential oils. Without wishing to be bound to theory, the presence of an essential oil provides the dosage form with a favorable essence.

In some embodiments, the composition is formulated as granules, powder, capsules, tablet, film, suspension, sachets, a chewing gum and suspension, preferably as a capsule, for example a gelatin capsule. In preferred embodiments, the gelatin capsule is a vegan based gelatin capsule.

The compositions disclosed herein include a sustained release composition or a combined sustained release fraction and immediate release fraction. Preferably, the therapeutic effect of the composition has a duration up to 10 hours, up to 11 hours, up to 12 hours, up to 14 hours, up to 16 hours or up to 18 hours.

In preferred embodiments, the composition disclosed herein comprises an immediate release (IR) fraction and a sustained release (SR) fraction, wherein the immediate release fraction comprises a therapeutically effective amount of cannabinoids and an edible oil; and wherein the sustained release fraction comprises a therapeutically effective amount of cannabinoids, and a mixture of emulsifiers which include 10-35% w/w monoglycerides and diglycerides, 0-5% w/w iota and/or kappa carrageenan and 60-90% w/w coconut oil.

In some embodiments of the composition, the total amount of cannabinoids in each dosage form is 1 mg to 100 mg, or 5 mg to 50 mg, preferably 6 mg to 40 mg, or 6 mg, 12 mg, 25 mg and 40 mg.

In a second aspect, provided herein is a method of treating a cannabinoid responsive disorder in a subject for at least 8 hours, comprising administering to the subject a therapeutically effect amount of an oral composition of cannabinoids comprising a range of 0% to 99% immediate release fraction and a range of 100% to 1% sustained release fraction, wherein each fraction independently comprises a cannabinoid and an excipient; wherein the excipient in the immediate release fraction is an edible oil and the excipient in the sustained release fraction comprises an emulsifier. Also provided is an oral composition of cannabinoids comprising a range of 0% to 99% immediate release fraction and a range of 100% to 1% sustained release fraction, wherein each fraction independently comprises a cannabinoid and an excipient; wherein the excipient in the immediate release fraction is an edible oil and the excipient in the sustained release fraction comprises an emulsifier, for use in treating a cannabinoid responsive disorder.

In some embodiments, the cannabinoid responsive disorder comprises attenuating a symptom of a disease, treat a disease or attenuating a side effect of a treatment wherein the cannabinoid responsive disorder is selected from disorders responsive to treatment with cannabis including but not limited to pain associated with cancer, neuropathic pain and HIV-associated sensory neuropathy; side effects of chemotherapy including nausea and pain; symptoms of neurology and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, sleep apnea, digestive diseases, collagen-induced arthritis, atherosclerosis and dystonia.

In some embodiments, the cannabinoid responsive disorder is cancer; pain associated with cancer; nausea associated with chemotherapy; and a combination thereof. In various embodiments, the composition exerts reduced hallucinatory effects in the subject when compared to smoking a cannabis containing cigarette or ingesting a cannabis containing foodstuff with the same amount of active ingredient.

In yet another aspect, provided herein is a method for the preparation of a combination sustained release (SR) and immediate release (IR) oral composition of a cannabinoid comprising the steps of
a. mixing a decarboxylated cannabinoid extract with an edible oil;
b. homogenizing the mixture of step a) at 80-120° C. to generate an IR fraction;
c. mixing a decarboxylated cannabinoid extract with an emulsifier and optionally an edible oil;
d. homogenizing the mixture of step c) at 80-120° C. to generate a SR fraction;
e. combining the IR fraction and the SR fraction;
   thereby preparing a combination sustained release and immediate release oral composition of a cannabinoid.

In some embodiments, a sustained oral composition of a cannabinoid is preferred and the process includes only steps c) and d).

In some embodiments of the methods, the method further includes the step of milling, drying, compressing or filling capsules, preferably filling gelatin capsules.

The preferred methods, uses, materials, and examples that will now be described are illustrative only and are not intended to be limiting; materials, uses and methods similar or equivalent to those described herein can be used in practice or testing of the invention. Other features and advantages of the invention will be apparent from the following figures, detailed description, and from the claims.

### BRIEF DESCRIPTION OF THE FIGURES

FIG. 1A-FIG. 1C show the efficacy and therapeutic window of the compositions disclosed herein in attenuating pain (FIG. 1A) and nausea (FIG. 1B) while concomitantly precluding the psychotropic effect (FIG. 1C) typically associated with cannabis.

### DETAILED DESCRIPTION OF THE INVENTION

The present inventors have now developed therapeutic compositions useful for sustained release or combined immediate and sustained release of cannabinoids. The therapeutic composition comprises a sustained release fraction or combined immediate and sustained release fractions. The sustained release fraction comprises an active pharmaceutical ingredient (i.e. cannabinoid extract or synthetic cannabinoid), an emulsifier selected from monoglyeride, diglyceride, carrageenan, lecithin, beeswax or any mixture thereof, and optionally an edible oil or a mixture of edible oils. The advantages of the composition over known cannabis compositions are manifold and include: (a) High bioavailability of the APIs in the composition.

(b) A flat pharmacokinetic profile that enables steady state level of beneficial effects for a duration of at least 10-12 hours.
(c) Avoidance of a sharp Cmax of APIs in the circulation and thus reduced level of undesirable side effects.
(d) Once per day easy administration regimen that promotes high patient compliance.

As used herein, the term “treatment” refers to therapeutic treatment of cannabinoid responsive disorder, wherein the object is to reduce or reverse the symptoms of the disorder. Those in need of treatment include those already experiencing the disease or condition, for example, spasticity in multiple sclerosis patients, pain in cancer patients or nausea in chemotherapy patients. The compositions or combinations disclosed herein are administered during or subsequent to the onset of the disease, symptom or condition. In some embodiments, treatment refers to prophylaxis, for example, prophylaxis of a disorder in a subject at risk of developing such a disorder, such as nausea in chemotherapy patients. Cₘₐₓ refers to the maximum (or peak) concentration (for example in the blood stream) that a drug achieves after the drug has been administered.

In some embodiments, a cannabinoid responsive disorder is selected from disorders responsive to treatment with cannabis including but not limited to pain associated with cancer, neuropathic pain and HIV-associated sensory neuropathy; side effects of chemotherapy including nausea and pain; symptoms of neurology and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, sleep apnea, digestive diseases, collagen-induced arthritis, atherosclerosis and dystonia.

The therapeutic window refers to the range of drug dosages of a medication that elicit a therapeutic response, without unacceptable adverse effects (toxicity), in a population of patients. Therapeutic window may also be referred to as “effective dose”. The therapeutic window of the present composition per dose form is about 1 mg to about 350 mg cannabinoid, about 5 mg to about 250 mg, about 5 mg to about 100 mg. Suitable dosage include about 5, 6.5, 7.5, 10, 12.5, 15, 17.5, 20, 22.5, 25, 27.5, 30, 32.5, 35, 37.5, 40, 42.5, 45, 47.5, 50, 52.5, 55, 57.5, 60, 62.5, 65, 67.5, 70, 72.5, 75, 77.5, 80, 82.5, 85, 87.5, 90, 92.5, 95, 97.5, 100, 102.5, 105, 107.5, 110 and up to 350 mg active ingredient (API), e.g. cannabinoid(s), in a sustained release or combined immediate release and sustained release composition. In some embodiments, each dosage form contains, for example, from about 0.1% to about 99.9% w/w, preferably from about 1% to about 60% w/w, of the active ingredient(s). In some embodiments, a dose unit includes 5 mg to 50 mg API, 6 mg to 40 mg API or dose units of 6 mg, 12 mg, 25 mg and 40 mg, which corresponds to 1.2%, 2.4%, 5% and 8% API in the formulation, respectively. Each dosage form or unit, may contain from about 50 mg to about 1000 mg, or about 100 to about 500 mg, or about 500 mg total composition, which includes sustained release or combined immediate release and sustained release fractions.

Pharmaceutical preparations for the combination therapy for enteral or parenteral administration are, for example, those in unit dosage forms, such as sugar-coated tablets, tablets, capsules or suppositories, or ampoules. If not indicated otherwise, these are prepared in a manner known per se, for example by means of conventional mixing, granulating, sugar-coating, dissolving or lyophilizing processes. It will be appreciated that the unit content of API per fraction contained in an individual dosage form need not in itself constitute an effective amount since the necessary effective amount can be reached by administration of a plurality of dosage units.

In some embodiments, the dosage form is a capsule or tablet. Capsule formulations may be a hard gelatin or soft gelatin type that contains the active API in solid, semi-solid, or liquid form. Gelatin capsules are formed from animal gelatin or synthetic or plant derived equivalents thereof. In some embodiments, the oral compositions disclosed herein are contained in a soft, vegetarian gelatin capsule.

As used herein, the singular forms “a”, “an” and “the” include plural forms unless the content clearly dictates otherwise.

### Cannabinoids

In some embodiments, the compositions disclosed herein provide one or more cannabinoids in an oral dosage form that can deliver to a subject a desired target PK profile, where the PK profile achieves a therapeutic level of active cannabinoids within a therapeutic window. Cannabinoids useful in the compositions disclosed are any member of a group of substances that are structurally related to tetrahydrocannabinol (THC) and that bind to a cannabinoid receptor such as CB1 or CB2 or both. The cannabinoid can be a naturally occurring compound (e.g. present in Cannabis), a compound metabolized by a plant or animal, or a synthetic derivative. The cannabinoid may be included in its free form, or in the form of a salt; an acid addition salt of an ester; an amide; an enantiomer; an isomer; a tautomer; a prodrug; a derivative of an active agent of the present invention; different isomeric forms (for example, enantiomers and diastereoisomers), both in pure form and in admixture, including racemic mixtures; enol forms. Various stereospecific enantiomers of cannabinoids are disclosed, for example, in U.S. Pat. Nos. 6,096,740 and 6,610,737.

In some embodiments, the cannabinoid(s) utilized in the present invention are a lipophilic concentrate of active cannabinoids achieved via CO2 extraction technique, and represents only one example of the different forms and extraction methods of cannabinoids useful for preparing the compositions disclosed herein. For example, U.S. Patent Application Publication 2005/0266108, hereby incorporated by reference in its entirety, describes a method of purifying cannabinoids obtained from plant material.

Cannabis sativa contains over 421 different chemical compounds, including over 60 cannabinoids. Cannabinoid plant chemistry is far more complex than that of pure THC, and different effects may be expected due to the presence of additional cannabinoids and other chemicals. Eighteen different classes of chemicals, including nitrogenous compounds, amino acids, hydrocarbons, carbohydrates, terpenes, and simple and fatty acids, contribute to the known pharmacological and toxicological properties of cannabis. (Huestis, M. (2007) Chem Biodivers. 4(8): 1770-1804).

The cannabinoids of the present invention can be any of a synthetic or natural 9-tetrahydrocannabinol (THC), 8-tetrahydrocannabinol, (+)-1,1-dimethylheptyl analog of 7-hydroxy-delta-6-tetrahydrocannabinol, cannabinol (CBN), cannabidivarin (CBDV), cannabidiolic acid (CBDA), cannabidiol (CBD), cannabichromene (CBC), cannabigerol (CBG), 3-(5′-cyano-1′,1′-dimethylpentyl)-1-(4-N-morpholinobutyryloxy) delta 8-tetrahydrocannabinol hydrochloride], dexanabinol, nabilone, levonantradol, or N-(2-hydroxyethyl)hexadecanoamide. The cannabinoids of the present invention can be any of the psychotropic or non-psychotropic cannabinoids. In preferred embodiments, the lipophilic mixture of cannabinoids comprises the following cannabinoid types and their derivatives (including their acidic and decarboxylated derivatives): Tetrahydrocannabinol (THC), Cannabidiol (CBD), Cannabigerol (CBG), Cannabichromene (CBC), Cannabinol (CBN), Cannabielsoin (CBE), iso-Tetrahydrocannabimol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV) and Cannabigerol Monomethyl Ether (CBGM).

Suitable amounts of API, e.g. cannabis extract, may be introduced and these amounts can be empirically determined using standard methods. The weight ratio of the cannabinoid extract to the excipients mixture may range from 1% to 99% w/w. Effective concentrations of individual dosage forms may range from 1% to 20% w/w which reflects about 5 mg to 100 mg, respectively.

### Oils

In some embodiments of a combined immediate release and sustained release composition, only the immediate release fraction comprises an edible oil. In other embodiments of a combined immediate release and sustained release composition, both the immediate release fraction and the sustained release fraction comprise an edible oil. In some embodiments the edible oil is a liquid, semi-solid or solid oil, preferably a liquid edible oil. In some embodiments, the oil is a vegetable, fruit, seed, nut or synthetic oil selected from coconut oil, wheat sprout oil, wheat germ oil, olive oil, sesame oil, peanut oil, almond oil, grape seed oil, palm oil, papaya seed oil, canola oil, sunflower oil, or a mixture thereof. The edible oil is preferably coconut oil or a mixture of coconut oil and another edible oil. Preferably, the edible oil is an organic edible oil, for example organic coconut oil and/or wheat sprout oil, for example organic wheat sprout oil.

### Excipients

The sustained release compositions disclosed herein include at least one emulsifier. The preferred emulsifier is selected from the group consisting of a monoglyceride, a diglyceride, beeswax, lecithin, a carrageenan and any mixture thereof. In some embodiments the composition includes an emulsifier in a concentration of 1% to 99% w/w. In preferred embodiments, the composition comprises an emulsifier at a concentration range of 5% to 80%, 10% to 20%, 10% to 35% or about 15% to 25% w/w.

In some embodiments, the emulsifier comprises a monoglyceride, a diglyceride or a mixture of a monoglyceride and a diglyceride. In some embodiments, the emulsifier comprises more than one monoglyceride and/or diglyceride.

The term “monoglyceride” or “acylglycerol” refers to a molecule with one glycerol moiety covalently bonded to a fatty acid chain via an ester bond.

The term “diglyceride” or “diacylglycerol” (DAG), refers to a molecule with one glycerol moiety covalently bonded to two fatty acid chains via ester bonds.

The monoglyceride, diglyceride or mixture of monoglyceride and diglyceride act as an emulsifier. A preferred emulsifier known in the art as “Glice” or “E471” is a mixture of monoglycerides and diglycerides that has gelling properties when mixed with oil, and forms a butter-like oil-gel.

In some embodiments the composition comprises a monoglyceride, a diglyceride or a mixture of a monoglyceride and a diglyceride at a concentration of 1% to 99%, 5% to 80%, 10% to 35%, 10% to 20%, or about 15% to 25% w/w.

In some embodiments, the emulsifier may comprise a polysaccharide. The polysaccharide may be linear or branched, sulfated or unsulfated. In some embodiments, the composition comprises one or more linear sulfated polysaccharide known as “carrageenan”.

The carrageenans are a family of linear sulfated polysaccharides that are extracted from edible seaweed and widely used in the food industry. The USPNF 23 describes carrageenan as hydrocolloid obtained by extraction and purification with water or aqueous alkali from few members of the class Rhodophyceae (red seaweed). It consists mainly of potassium, sodium, calcium magnesium and ammonium sulfate esters of galactose and 3,6-anhydrogalactose copolymers. These hexoses are alternatively linked at the α-1,3 and β-1,4 sites in the polymer.

The carrageenans are divided into three families according to the position of sulfate groups and the presence of anhydrogalactose. Lambda-carrageenan (λ-carrageenan) is a nongelling polymer containing about 35% ester sulfate by weight and no 3,6-anhydrogalactose. Iota-carrageenan (t-carrageenan) is a gelling polymer containing about 32% ester sulfate by weight and approximately 30% 3,6-anhydrogalactose. Kappa carrageenan (κ-carrageenan) is a strongly gelling polymer which has a helical tertiary structure that allows gelling. It contains 25% ester sulfate by weight and approximately 34% 3,6-anhydrogalactose. Among the three carrageenans, λ-carrageenan is the only nongelling polymer.

In some embodiments the composition comprises a carrageenan or a mixture of a carrageenans at a concentration of 0.01% to 10% w/w, preferably at a concentration of 0.01% to about 5% or 1% to about 7% w/w.

In some embodiments, the composition includes lecithin. In some embodiments, the composition further includes lecithin.

Lecithin is a generic term to designate any group of fatty substances occurring in animal and plant tissues that are composed of phosphoric acid, choline, fatty acids, glycerol, glycolipids, triglycerides, and phospholipids (e.g., phosphatidylcholine, phosphatidylethanolamine, and phosphatidylinositol).

For example, soybean-derived lecithin dietary supplements are composed of 19-21% phosphatidylcholine, 8-20% phosphatidylethanolamine, 20-21% inositol phosphatides, 33-35% soybean oil, 2-5% sterols, 5% carbohydrates/free, 1% moisture, and 5-11% other phosphatides.

Lecithin has low solubility in water, but is an excellent emulsifier. In aqueous solution, its phospholipids can form either liposomes, bilayer sheets, micelles, or lamellar structures, depending on hydration and temperature. This results in a type of surfactant that usually is classified as amphipathic. Lecithin is sold as a food supplement and for medical uses. In some embodiments the composition comprises Lecithin at a concentration of 0% to 10% w/w, preferably at a concentration of about 2% to 8% w/w or about 5% w/w.

In some embodiments, the composition includes beeswax. In some embodiments, the composition further includes beeswax. Beeswax is a natural wax produced in the bee hive of honey bees of the genus Apis. It consists mainly of fatty acid esters and long-chain alcohols. Beeswax has food, cosmetic and pharmaceutical applications. For example, beeswax known as food additive E901, is used in small quantities as a glazing agent to prevent water loss, or used to provide surface protection for some fruits. Soft gelatin capsules and tablet coatings may also incorporate E901.

In some embodiments the composition comprises a beeswax at a concentration of 0% to 10% w/w, preferably at a concentration of about 1% w/w.

Therefore, an emulsifier selected from λ-carrageenan, κ-carrageenan, ι-carrageenan, monoglyceride, diglyceride, lecithin, beeswax or any mixture thereof, when formulated with a cannabinoid or cannabinoid extract, and optionally an edible oil and/or one or more pharmaceutical excipient provides sustained or extended release of cannabinoids.

Depending on the dosage form, the one or more optional pharmaceutical excipient may be selected from a diluent, a binder, a lubricant, a disintegrant, a flavoring agent, a coloring agent, a stabilizer, a surfactant, a glidant, a plasticizer, a preservative, essential oil and a sweetener. A person skilled in the art will be able to select the best excipient or mixture of excipients for the desired formulation. Each excipient may fall within one or more classifications.

A diluent may be selected from, for example, calcium carbonate, calcium phosphate dibasic, calcium phosphate tribasic, calcium sulfate, microcrystalline cellulose, microcrystalline silicified cellulose, powdered cellulose, dextrate, dextrose, fructose, lactitol, lactose anhydrous, lactose monohydrate, lactose dihydrate, lactose trihydrate, mannitol, sorbitol, starch, pregelatinized starch, sucrose, talc, xylitol, maltose, maltodextrin, maltitol.

A binder may be selected from, for example, acacia, alginic acid, carbomer, carboxymethylcellulose calcium, carbomethylcellulose sodium, microcrystalline cellulose, powdered cellulose, ethyl cellulose, gelatin liquid glucose, guar gum, hydroxyethyl cellulose, hydroxypropyl cellulose, hydroxypropylmethyl cellulose, maltodextrin, methylcellulose, polydextrose, polyethtylene oxide, povidone, sodium alginate, starch paste, pregelatinized starch, sucrose, tragacanth, low-substituted hydroxypropyl cellulose, glucose, sorbitol.

A suitable filler may be selected from, for example, starch derivatives, such as corn starch, potato starch or rice starch, polysaccharides such as dextrins, maltodextrins, dextrates, microcrystalline cellulose, powdered cellulose, mixture of microcrystalline cellulose and guar gum, coprocessed blends of microcrystalline cellulose; and polyhydric alcohols, such as xylitol and sorbitol.

A disintegrant may be selected from, for example, alginic acid, carbon dioxide, carboxymethylcellulose calcium, carboxymethylcellulose sodium, microcrystalline cellulose, powdered cellulose, croscarmelose sodium, crospovidone, sodium docusate, gaur gum, hydroxypropyl cellulose, methylcellulose, polacrilin potassium, poloxamer, povidone, sodium alginate, sodium glycine carbonate, sodium lauryl sulfate, sodium starch glycolate, starch, pregelatinized starch, low-substituted hydroxypropyl cellulose.

A glidant may be selected from, for example, calcium silicate, powdered cellulose, starch, talc, colloidal silicon dioxide.

A lubricant may be selected from, for example, magnesium stearate, stearic acid, sodium stearyl fumarate, magnesium lauryl sulphate, talc, polyethylene glycol, and glyceryl behenate.

A suitable essential oil may be selected from Bergamot oil (extracted from Citrus aurantium L. subsp. bergamia Wright et Arn.); Ylang ylang oil (extracted from Cananga odorata Hook. f. and Thoms.); Jasmine essential oil (extracted from Jasminum officinale L.). In one embodiment, a mixture of essential oils comprises equal portions totaling about 0.01% to about 1% w/w, preferably about 0.1% w/w of the total composition. Other essential oils are possible.

A suitable sweetener may be selected from sugars such as sucrose, lactose and glucose; cyclamate and salts thereof; saccharin and salts thereof; and aspartame.

A flavouring agent may be selected from natural or synthetic flavours such as, for example, strawberry flavour, wild cherry flavour, green apple flavour, spearmint flavour and peppermint flavour.

In various embodiments, one or more of the ingredients of the composition is an organic ingredient.

In a further optional embodiment of the invention, the sustained release oral composition further comprises a coating. The coating material may be selected from materials known to a person skilled in the art.

Further disclosed herein is a sustained release oral formulation of cannabinoid wherein the dissolution profile of the oral composition releases 20% to 50% in 2 hours and greater than 80% in more than 12 hours of the cannabinoid content of the formulation.

### Indications

The compositions disclosed herein are beneficial in treating and/or reducing the symptoms of a variety of diseases and disorders responsive to treatment with cannabis including but not limited to pain associated with cancer, neuropathic pain and HIV-associated sensory neuropathy; side effects of chemotherapy including nausea; symptoms of neurology and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, sleep apnea, digestive diseases, collagen-induced arthritis, atherosclerosis and dystonia.

### Administration

Although the cannabis extract disclosed herein can be formulated for different modes of administration the preferred formulation is an oral formulation for immediate release, sustained release or a combination of immediate release and sustained release.

Persons skilled in the art are aware of the best modes of administration for cannabinoids. The useful dosage to be administered and the particular mode of administration will vary depending upon such factors as the age, weight and the particular subject, the therapeutic or diagnostic use contemplated, and the form of the formulation.

The “therapeutically effective dose” as used herein is thus determined by such considerations as are known in the art. The dose must be effective to achieve improvement including but not limited to improved survival rate or more rapid recovery, or improvement or elimination of symptoms and other indicators as are selected by those skilled in the art.

Provided herein are compositions and methods for treating cannabinoid responsive diseases and disorders by administering an effective amount of a sustained release or combined immediate release and sustained release composition to a subject in need thereof. An effective amount is an amount sufficient to eliminate or to alleviate symptoms. “Sustained release” means that the active cannabinoids are released from the composition over time so that their plasma concentration is maintained within the therapeutic window (above the therapeutically minimal effective concentration but below toxic levels) over an extended period of time. Non formal POC studies in the clinic of the formulations described herein have surprisingly found that the duration of therapeutic effect is longer than expected. The therapeutic effect of a 25 mg combined immediate and sustained release dose lasts up to about 12 hours and the therapeutic effect of a 40 mg combined immediate and sustained release dose lasts up to about 16 hours. The dosage form is preferably administered once per day, for example in the morning, with an optional additional administration in the afternoon, evening or night to achieve a complete 24 hour coverage of the beneficial therapeutic effects.

### Manufacture Method

Yet another embodiment of the present invention is related to the process for the preparation of sustained release oral formulation of cannabinoid extract, for use per se or in a combined immediate release and sustained release composition. The method for the preparation of a combination sustained release (SR) and immediate release (IR) oral composition of a cannabinoid includes the steps of
a. mixing a decarboxylated cannabinoid extract with an edible oil;
b. homogenizing the mixture of step a) at 80-120° C. to generate an IR fraction;
c. mixing a decarboxylated cannabinoid extract with an emulsifier and optionally an edible oil;
d. homogenizing the mixture of step c) at 80-120° C. to generate a SR fraction;
e. combining the IR fraction and the SR fraction
   thereby preparing a combination sustained release and immediate release oral composition of a cannabinoid.

In some embodiments, a sustained oral composition of a cannabinoid is preferred and the process for preparing such sustained release oral composition of a cannabinoid includes only steps c) and d) from above, as follows:
a. mixing a decarboxylated cannabinoid extract with an emulsifier and optionally an edible oil;
b. homogenizing the mixture of step c) at 80-120° C. to generate a SR fraction; thereby preparing a sustained release oral composition of a cannabinoid.

In some embodiments of the methods, the method further includes the step of milling, drying, compressing or filling capsules, preferably filling gelatin capsules.

The dosage form of the present invention may be prepared using conventional techniques employed in the art for mixing, compaction, granulation, milling, drying, compressing and/or filling in capsules. The oral formulation may be selected from sprinkle granules or powder for reconstitution in a suspension, tablet, soluble tablet, rapidly disintegrating tablet, orally disintegrating tablet, rapidly disintegrating film, orally disintegrating powder for capsules, suspension or sachets, effervescent tablet, a chewable tablet, water dispersible tablet, orodispersible tablet, a chewing gum and suspension.

The individual dosage forms may be packaged together in the form of a kit, optionally with instructions for use. Alternatively, the individual dosage forms may be individually packaged, as in foil envelopes or in a blister pack.

Where aspects or embodiments of the invention are described in terms of Markush groups or other grouping of alternatives, those skilled in the art will recognize that the invention is also thereby described in terms of any individual member or subgroup of members of the group.

The aspects and embodiments provided herein have been described in an illustrative manner, and it is to be understood that the terminology used is intended to be in the nature of words of description rather than of limitation.

Many modifications and variations are possible in light of the above teachings. It is, therefore, to be understood that within the scope of the appended claims, the invention can be practiced otherwise than as specifically described.

Throughout this application, various publications, including United States patents, are referenced by author and year and patents by number. The disclosures of these publications and patents and patent applications in their entireties are hereby incorporated by reference into this application in order to more fully describe the state of the art to which this invention pertains.

The present disclosure is illustrated in detail below with reference to examples, but is not to be construed as being limited thereto.

Citation of any document herein is not intended as an admission that such document is pertinent prior art, or considered material to the patentability of any claim of the present disclosure. Any statement as to content or a date of any document is based on the information available to applicant at the time of filing and does not constitute an admission as to the correctness of such a statement.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

### Example 1

### Preparation of the Cannabinoids Extract

1. The lipophilic active cannabinoid ingredients were extracted from raw botanical plant material by CO₂ extraction. Other extraction methods known in the art are acceptable.
2. A decarboxylation step was carried out by heating the extract to 119° C. (possible range 110° C. to 140° C.) for 30 minutes (possible range about 20 to 40 minutes). The heated extract layer was no more than 3-4 mm deep to ensure homogeneous temperature and decarboxylation process. The result of this process is referred to as the “cannabinoids extract”.

### Example 2

### Preparation of Dosage Forms Comprising Sustained Release or Combined Immediate Release and Sustained Release Formulations

The following tables provide exemplary, non-limiting, compositions comprising a sustained release or combined immediate release and sustained release fractions in one dosage form.

TABLE A
Formula A - immediate release formulation

dose

| ingredient | 6 mg | 12 mg | 25 mg | 40 mg |
|---|---|---|---|---|
| Organic grape | 10% | 10% | 10% | 10% |
| seed oil w/w | | | | |
| Organic sprout | 8.8% | 7.6% | 5.0% | 2.0% |
| wheat oil w/w | | | | |
| Organic Coconut | 80% | 80% | 80% | 80% |
| oil w/w | | | | |
| Decarboxylated | 1.2% | 2.4% | 5.0% | 8.0% |
| total Cannabinoid | | | | |
| CO₂ extract w/w | | | | |

TABLE B
Formula B - Sustained release formulation

dose

| ingredient | 6 mg | 12 mg | 25 mg | 40 mg |
|---|---|---|---|---|
| Mono and | 15.0% | 15.0% | 15.0% | 15.0% |
| diglycerides w/w | | | | |
| Carrageenan Iota | 1.0% | 1.0% | 1.0% | 1.0% |
| w/w | | | | |
| Lecithin w/w | 5% | 5% | 5% | 5% |
| Organic Coconut | 77.8% | 76.6% | 74.0% | 71.0% |
| oil w/w | | | | |
| Decarboxylated | 1.2% | 2.4% | 5.0% | 8% |
| total Cannabinoid | | | | |
| CO₂ extract w/w | | | | |

### Immediate Release (IR) Formulation

1. The cannabinoids extract was diluted with an oil mixture preheated to 100° C. (possible range −80° C. to 120° C.), according to Table A, comprising of (percentage by weight of ingredients):
a. Organic coconut oil
b. Organic Sprout wheat oil
c. Extra virgin organic olive oil or grape seed oil
2. The mixture was homogenized by using a heavy-duty lab grade stirrer while heating to keep the same temperature for about 20 minutes (possible range is 10 to 30 minutes).
3. The oil and cannabis extract mixture cooled down to room temperature while stirring.

A mixture of essential oils described below was added (optional), while stirring.

A mixture of essential oils comprised equal portions totaling 0.1% of total mixture: Bergamot oil (extracted from Citrus aurantium L. subsp. bergamia Wright et Arn.); Ylang oil (extracted from Cananga odorata Hook. f. and Thoms.); Jasmine essential oil (extracted from Jasminum officinale L.).

4. The resulting mixture was termed “Formulation A”.

### Sustained Release (SR) Formulation

1. The cannabinoids extract was diluted with a mixture below preheated to 100° C. (possible range −80° C. to 120° C.), according to Table B comprising of (percentage by weight of ingredients):
a. Organic coconut oil
b. Mono and Diglycerides emulsifying powder (E471)
c. Iota Carrageenan powder
d. Lecithin

The mixture was homogenized using a heavy-duty lab grade stirrer while heating to keep the same temperature for 20 minutes (possible range about 10 to 30 minutes).

3. The resulting mixture was termed “Formulation B”.

### Capsule Filling

Each capsule contained a total of 500 mg of formulation, including up to a total of 350 mg API.

1. Each capsule may contain any proportion of the IR (Formula A) and SR (Formula B) formulations, for example:
a. 0% A-100% B=pure SR capsules
b. 90% A-10% B=primarily IR capsules
c. 50% A-50% B=IR+SR combination capsules (in equal amounts)
2. The lower part of the capsule was filled with the desired amount of formulation A first.
3. The capsules were frozen to congeal formulation A.
4. The remaining capsule space was filled with the desired amount of formulation B.
5. The capsules were closed and cooled until they reached room temperature.

### Example 3

### Proof of Concept Study for Palliative Oncology Patients

The study included 3 study arms, with five patients per arm. The arms were as follows:
1. Combined immediate release and sustained release capsule for oral administration, 25 mg active ingredient. Single daily administration in the morning, after a light breakfast.
2. A capsule containing raw ground cannabis flowers, 25 mg active ingredients Single daily administration in the morning, after a light breakfast.
3. Raw cannabis flowers smoked in a cigarette, 25 mg active ingredients. Smoking was allowed throughout the day, at any time of need.

The study endpoints (measured via reports and questionnaires):

1. Reduction in pain
2. Elimination of nausea
3. Euphoric effect—mood enhancement.

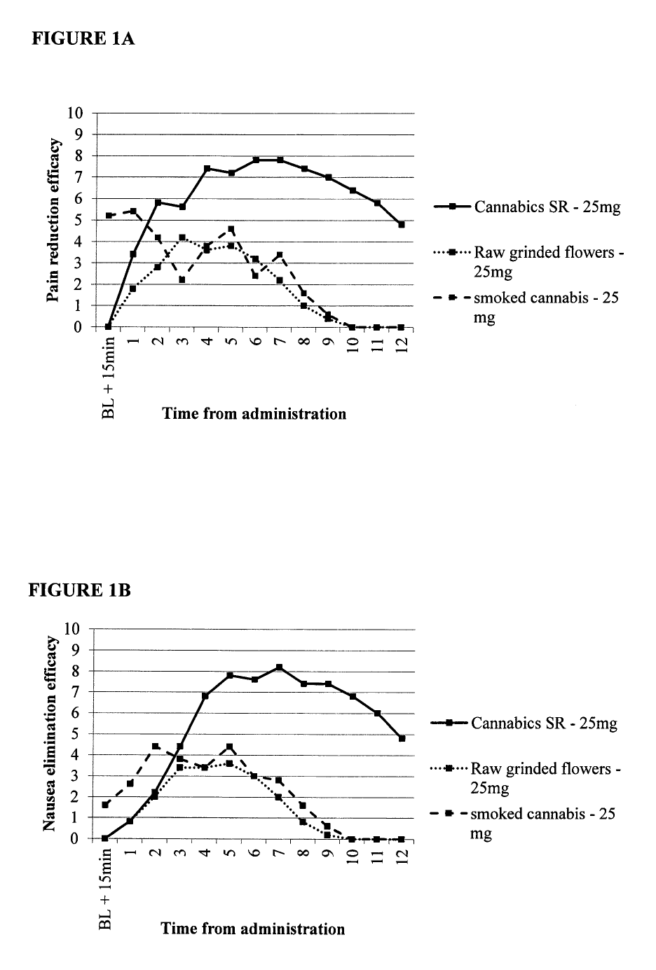

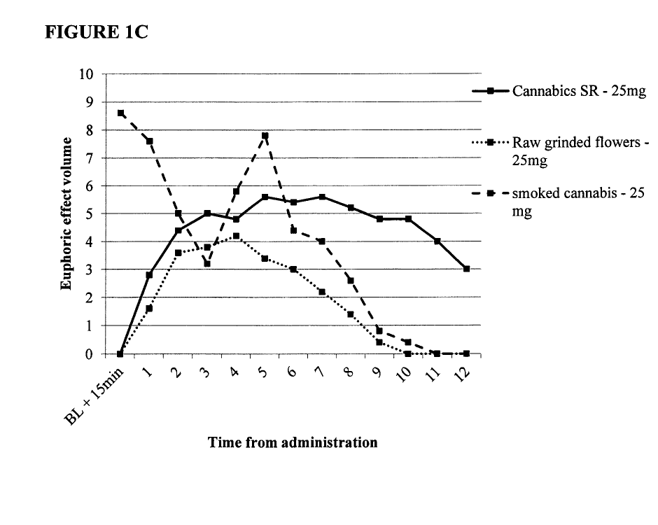

FIGS. 1A-IC provide data showing relative pain reduction, elimination of nausea and mood enhancement for patients over twelve hours. FIG. 1A shows that the composition of the invention comprising 25 mg active cannabinoids (IR and SR) effectively reduced pain in patients for more than 12 hours. The pain reduction effect was much more dramatic and lasted much longer than the effect provided by the smoked cannabis or raw ground cannabis flowers. FIG. 1B shows that the composition of the invention comprising 25 mg active cannabinoids (IR and SR) effectively reduced nausea in patients for more than 12 hours. The nausea reduction effect was much more dramatic and lasted much longer than the effect provided by the smoked cannabis or raw ground cannabis flowers. FIG. 1C shows that the composition of the invention comprising 25 mg active cannabinoids (IR and SR) had a moderate psychoactive effect in patients for about 10 hours. The psychoactive effect was lower than the effect provided by the smoked cannabis and never peaked, providing a reduced and stable euphoric effect.

### Example 4

### IR, SR and Combined Compositions

Tables C and D provide examples of sustained release (SR) compositions according to the invention. The SR compositions are used to prepare sustained release compositions or combined sustained release and immediate release compositions.

TABLE C
SR formulation C

Dose

| Ingredient | 6 mg | 12 mg | 25 mg | 40 mg |
|---|---|---|---|---|
| Mono and | 98.8% | 97.6% | 95% | 92% |
| diglycerides | | | | |
| (w/w) | | | | |
| Decarboxylated | 1.2% | 2.4% | 5% | 8% |
| total | | | | |
| Cannabinoid | | | | |
| CO₂ extract | | | | |
| (w/w) | | | | |

### Sustained Release (SR) Formulation C

1. The cannabis extract is diluted with Mono and Diglycerides emulsifying powder (E471) in the amounts presented in Table C
2. The mixture is homogenized using a heavy-duty lab grade stirrer while heating to keep the same temperature for 20 minutes (possible range about 10 to 30 minutes).

### Capsule Filling

Each capsule contains a total of 50 to 1000 mg, or about 500 mg of composition (SR or IR+SR), and up to a total of 350 mg API.

1. Each capsule contains any proportion of the IR (for example, Formula A) and SR (Formula C) formulations, for example:
a. 0% A-100% C=pure SR capsules
b. 90% A-10% C=primarily IR capsules
c. 50% A-50% C=IR+SR combination capsules (in equal amounts)
2. The lower part of the capsule is filled with the desired amount of formulation A first.
3. The capsules are frozen to congeal formulation A.
4. The remaining capsule space is filled with the desired amount of formulation C.
5. The capsules are closed and cooled until they reach room temperature.

TABLE D
SR formulation D

dose

| ingredient | 6 mg | 12 mg | 25 mg | 40 mg |
|---|---|---|---|---|
| Mono and | 78.8% | 77.6% | 75% | 72% |
| diglycerides w/w | | | | |
| Lecithin w/w | 20% | 20% | 20% | 20% |
| Decarboxylated | 1.2% | 2.4% | 5% | 8% |
| total Cannabinoid | | | | |
| CO₂ extract w/w | | | | |

### Sustained Release (SR) Formulation D

1. The cannabis extract is diluted with Mono and Diglycerides emulsifying powder (E471) in the amounts presented in Table D. Lecithin is added.
2. The mixture is homogenized using a heavy-duty lab grade stirrer while heating to keep the same temperature for 20 minutes (possible range about 10 to 30 minutes).

### Capsule Filling

Each capsule contains a total of 50 to 1000 mg, or about 500 mg of composition (SR or IR+SR), and up to a total of 350 mg API.

1. Each capsule contains any proportion of the IR (for example, Formula A) and SR (Formula D) formulations, for example:
a. 0% A-100% D=pure SR capsules
b. 90% A-10% D=primarily IR capsules
c. 50% A-50% D=IR+SR combination capsules (in equal amounts)
   2. The lower part of the capsule is filled with the desired amount of formulation A first.
   3. The capsules are frozen to congeal formulation A.
   4. The remaining capsule space is filled with the desired amount of formulation D.
   5. The capsules are closed and cooled until they reach room temperature.

The exemplary formulations described above in Tables A-D, are prepared using for example, the methods described herein.

The inventions illustratively described herein may suitably be practiced in the absence of any element or elements, limitation or limitations, not specifically disclosed herein. The terms “comprising”, “having,” “including,” containing”, etc. shall be construed without limitation (e.g., meaning “including, but not limited to,”). Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise indicated.

Although the above examples have illustrated particular ways of carrying out embodiments of the invention, in practice persons skilled in the art will appreciate alternative ways of carrying out embodiments of the invention, which are not shown explicitly herein. It should be understood that the present disclosure is to be considered as an exemplification of the principles of this invention and is not intended to limit the invention to the embodiments illustrated.

Those skilled in the art will recognize, or be able to ascertain using no more than routine experimentation, equivalents of the specific embodiments of the invention described herein. Such equivalents are intended to be encompassed by the following claims.

1. An oral composition of cannabinoids comprising a range of 0% to 99% immediate release fraction and a range of 100% to 1% sustained release fraction, wherein each fraction independently comprises a cannabinoid and an excipient; wherein the excipient in the immediate release fraction is an edible oil and the excipient in the sustained release fraction comprises an emulsifier.

2. The oral composition of claim 1, wherein the cannabinoid comprises a lipophilic mixture of cannabinoids that contains at least one of the following: Tetrahydrocannabinol (THC), Cannabidiol (CBD), Cannabigerol (CBG), Cannabichromene (CBC), Cannabinol (CBN), Cannabielsoin (CBE), iso-Tetrahydrocannabimol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV) and Cannabigerol Monomethyl Ether (CBGM) and derivatives thereof.

3. The oral composition of claim 2, wherein the emulsifier is selected from a group consisting of a monoglyceride, a diglyceride, beeswax, lecithin, a carrageenan and any mixture thereof.

4. The oral composition of claim 3, wherein the emulsifier comprises a mixture of monoglycerides and diglycerides.

5. The oral composition of claim 4, wherein the emulsifier comprises the carrageenan.

6. The oral composition of claim 5, wherein the carrageenan is selected from the group consisting of lambda-carrageenan, kappa-carrageenan, iota-carrageenan and any mixture of carrageenans.

7. The oral composition of claim 3, wherein the emulsifier is present in a concentration range of 1% to 99%, 5% to 80%, 10% to 35%, 10% to 20%, or about 15%-25%% w/w.

8. The oral composition of claim 7, wherein the emulsifier comprises a mixture of monoglyceride and diglyceride at a total concentration of 1% to 99% w/w and the carrageenan or mixture of carrageenans at a total concentration of 0.01% to 10% w/w.

9. The oral composition of claim 3, wherein the sustained release fraction further comprises the edible oil.

10. The oral composition claim 9, wherein the edible oil in the immediate release fraction and in the sustained release fraction are independently selected from coconut oil, wheat sprout oil, olive oil, sprouted wheat oil, sesame oil, peanut oil, grape seed oil, palm oil, papaya seed oil or any combination thereof.

11. The oral composition of claim 10, wherein the edible oil in the immediate release fraction and in the sustained release fraction comprises the coconut oil.

12. The oral composition of claim 1, wherein the cannabinoid is present in a range of 1 mg to 350 mg active cannabinoid, preferably 5 mg to 100 mg, or 6 mg to 40 mg.

13. The oral composition of claim 12, further comprising at least one excipient selected from a group consisting of a diluent, a binder, a lubricant, a disintegrant, a flavoring agent, a coloring agent, a stabilizer, a surfactant, a glidant, a plasticizer, a preservative, an essential oil and a sweetener.

14. The oral composition of claim 1, wherein therapeutic effect of the oral composition has a duration from 6 to 18 hours.

15. The oral composition of claim 1, the oral composition comprising an immediate release (IR) fraction and a sustained release (SR) fraction, wherein the immediate release fraction comprises a therapeutically effective amount of cannabinoids and the edible oil; and wherein the sustained release fraction comprises the therapeutically effective amount of cannabinoids, 10-35% monoglycerides and diglycerides, 0-5% iota carrageenan and 60-90% coconut oil.

16. The oral composition of claim 1, wherein cannabinoids are present in a total amount 6 mg to 40 mg per dosage form.

17. The oral composition of claim 1, formulated as a gelatin capsule.

18. A method of treating a cannabinoid responsive disorder for at least 8 hours in a subject, comprising administering to the subject a therapeutically effect amount of the oral composition of claim 1.

19. The method of claim 18, wherein the cannabinoid responsive disorder selected from a group consisting of cancer; pain associated with the cancer; nausea associated with chemotherapy; and a combination thereof.

20. The method of claim 18, wherein the oral composition exerts a reduced hallucinatory effects in the subject when compared to smoking a cannabis containing cigarette or ingesting the cannabis containing foodstuff with the same amount of active ingredient.

21. A process for preparation of combination sustained release (SR) and immediate release (IR) oral composition of a cannabinoid, comprising steps of
a. mixing a decarboxylated cannabinoid extract with an edible oil;
b. homogenizing a mixture of step a) at 80-120° C. to generate an IR fraction;
c. mixing the decarboxylated cannabinoid extract with an emulsifier and optionally the edible oil;
d. homogenizing the mixture of step c) at 80-120° to generate a SR fraction;
e. combining the IR fraction and the SR fraction
thereby preparing a combination sustained release and immediate release oral composition of the cannabinoid.

22. The process of claim 21, further comprising a step of milling, drying, compressing or filling capsules, preferably filling capsules.
